(19) United States
(12) Patent Application Publication
(10) Pub. No.: US 2007/0014190 A1
(43) Pub. Date: Jan. 18, 2007

(54) Multi-level pulser for an ultrasound system

(76) Inventors: Keith Alan Fehl, Calendonia, WI (US); Yoichi Suzuki, Tokyo (JP)

Correspondence Address: THE SMALL PATENT LAW GROUP LLP, 611 OLIVE STREET, SUITE 1611, ST. LOUIS, MO, 63101, US

(21) Appl. No.: 11/181,514

(22) Filed: Jul. 14, 2005

(51) Int. Cl. H04B 1/02; A61B 8/00

(52) U.S. Cl. 367/138; 600/437

(57) ABSTRACT

A multi-level pulser for an ultrasound system is provided that receives a reference voltage input signal, a shading signal, and a control signal. The pulser produces an output signal for driving a transducer, with the output signal based on the reference voltage signal, the shading signal, and the control signal. The output signal includes a continuous wave (CW) signal interleaved with a pulse wave (PW) signal. The PW signal may have multiple levels of amplitude.

### BACKGROUND OF THE INVENTION

This invention relates generally to ultrasound systems, and more particularly, to a pulser component of a beamformer (BF) for an ultrasound system.

Known beamformers (BFs) typically use digital custom integrated circuit (IC) chips to perform the functions of the beamformer associated with signals transmitted to and received from transducer elements of an ultrasound probe. An application specific IC (ASIC) may be used within the pulser to generate signals to a transducer to generate ultrasound wave forms for scanning a patient. The transducer elements may generate wave forms, for example, for pulsed ultrasound or continuous-wave ultrasound. Pulsed wave (PW) ultrasound produces cycles of ultrasound waves separated in time with gaps of no ultrasound waves. Pulsed ultrasound may be used to provide higher quality anatomic detail in ultrasound images. PW devices are able to select information from a particular depth along the ultrasound beam. Continuous wave (CW) ultrasound produces continuous cycles of ultrasound waves over time. CW devices may produce Doppler images with broad detectable tissue or blood flow motion in the image. An ultrasound system may include a pulser component for producing PW signals and a pulser component for producing CW signals. Both PW and CW pulser components are often combined into one multipurpose ultrasound system.

Typically, an ultrasound system may be capable of producing PW and CW spectral Doppler, as well as color Doppler. However, the cost of such systems having multiple modes of imaging is higher due to the complexity of the pulsers and receivers for transmitting ultrasound waves and receiving ultrasound echoes for the various modes. The increased cost is due in part to the increased complexity and cost of the associated beamformers.

### BRIEF DESCRIPTION OF THE INVENTION

In one embodiment, a multi-level pulser for an ultrasound system is provided that receives a reference voltage input signal, a shading signal, and a control signal. The pulser produces an output signal for driving a transducer, with the output signal based on the reference voltage signal, the shading signal, and the control signal. The output signal includes a continuous wave (CW) signal interleaved with a pulse wave (PW) signal. The PW signal may have multiple levels of amplitude.

In another embodiment, a method is provided for producing a pulser signal to an ultrasound system transducer. The method includes receiving a reference voltage signal for a first input, receiving a shading signal for a second input, and receiving a control signal for a third input. The method further includes producing an output signal for driving a transducer, with the output signal based on the reference voltage signal, the shading signal, and the control signal. The output signal comprises a continuous wave (CW) signal interleaved with a pulse wave (PW) signal.

### BRIEF DESCRIPTION OF THE DRAWINGS

FIG. 1 is a block diagram of an ultrasound system pulser constructed in accordance with an embodiment of the present invention.

FIG. 2 is a diagram illustrating an output signal from a pulser formed in accordance with an embodiment of the present invention.

FIG. 3 is a detailed block diagram of the pulser of FIG. 1.

FIG. 4 is a flowchart of a method of producing an output signal in accordance with an embodiment of the present invention.

FIG. 5 is a detailed block diagram of the field effect transistor (FET) circuitry of the pulser of FIG. 3.

FIG. 6 is a detailed block diagram of the pulser ASIC of the pulser of FIG. 3.

### DETAILED DESCRIPTION OF THE INVENTION

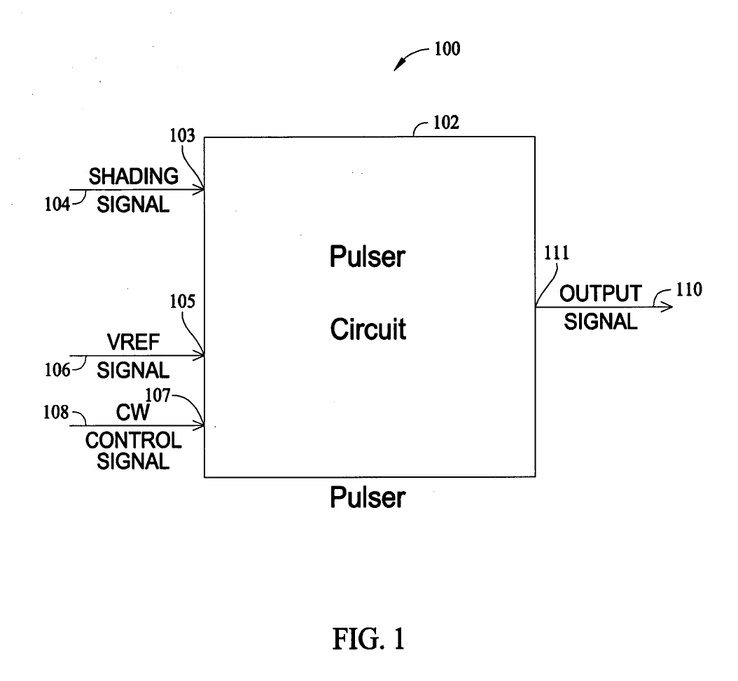

FIG. 1 is a block diagram of an ultrasound system pulser 100 constructed in accordance with an embodiment of the present invention. A pulser circuit 102 receives at first, second, and third inputs 103, 105, and 107 corresponding signals, for example, a shading signal 104, a voltage reference (VREF) 106 signal, and a continuous wave (CW) control signal 108. Other inputs may be present, for example, clocking inputs (not shown). Based on the three inputs 103, 105, and 107, an output signal 110 (e.g., beamforming signal) is produced on an output 111 and provided to an ultrasound transducer (not shown in FIG. 1) as is known. In an exemplary embodiment, and as described herein, the output signal 110 includes a continuous wave (CW) signal time-interleaved with a pulse wave (PW) signal. The interleaving of the CW and PW signals allows the transducer to correspondingly transmit CW and PW waveforms concurrently, resulting in concurrent acquisition of, for example, CW Doppler and PW Doppler echo data.

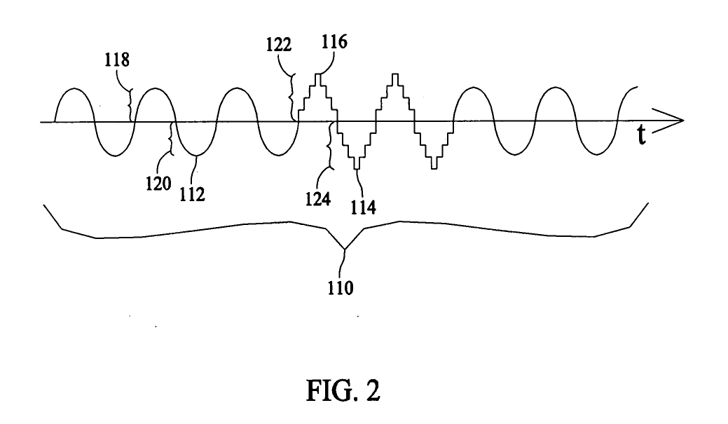

FIG. 2 is a diagram illustrating the output signal 110. A CW signal 112 is time-interleaved with a PW signal 114 to form the output signal 110. The CW signal 112 includes a positive portion 118 and a negative portion 120, and the PW signal 114 includes a positive portion 122 and a negative portion 124. The PW signal 114 is shown as having multiple levels of magnitude, for example, level 116. The pulser 100 produces a CW signal 112 interleaved with a multi-level PW signal 114. The interleaving of signals may be periodic or non-periodic.

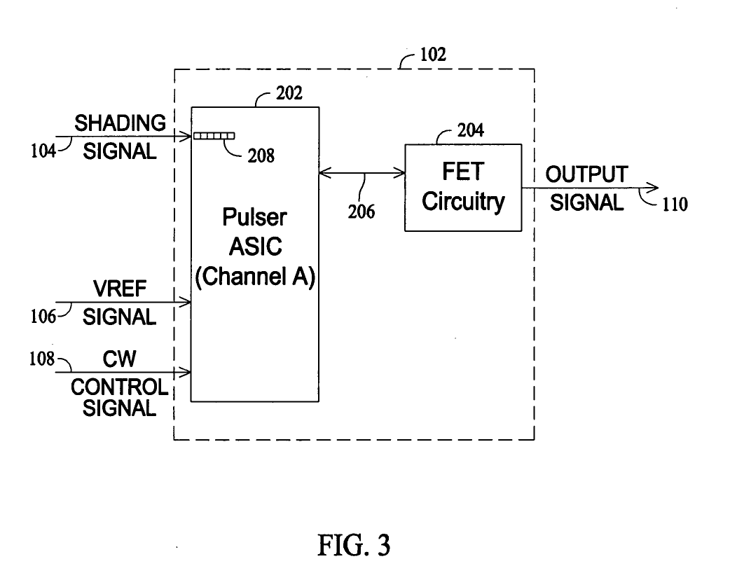

FIG. 3 is a block diagram illustrating the components of the pulser 100 of FIG. 1. The pulser circuit 102 is configured to and capable of producing ultrasound transmit signals of a multi-level PW pulser and a CW pulser. A pulser ASIC 202 receives and multiplies the shading signal 104 and the VREF signal 106 to produce a setting for the magnitude of the output signal 110 (e.g., signal output level). The setting may be provided as an input to a pair of CW and PW drivers (not shown) of the ASIC 202. In an exemplary embodiment, the shading signal 104 is received as a six bit word 117, with values of the six bit word 117 designating a plurality of levels of amplitude for the PW signal 114 (shown in FIG. 2). For at least one value of the six bit word 117, all bits are set to ones or highs, with the value of the VREF 106 signal used to establish a maximum value for the output signal 110. Other values of the six bit word 117 modify or adjust the magnitude of the VREF signal 106 and results in the multiple levels 116 (shown in FIG. 2), which are lesser than the maximum level. The magnitude of the output signal 110 is thereby modified or adjusted. Based on the value of the CW control signal 108, one driver of the pair of CW and PW drivers, is active. When the CW driver is active, a CW signal 112 (shown in FIG. 2) forms the output signal 110. When the PW driver is active, a PW signal 114 forms the output signal 110.

The field effect transistor (FET) circuitry 204 is connected to the ASIC 202 via an interface 206 (e.g., communication link). In an exemplary embodiment, the FET circuitry 204 is external to the ASIC 202 and provides the output signal 110. Thus, using the signals at the interface 206 and high voltage (HV) supplies (not shown), the FET circuitry 204 produces the output signal 110.

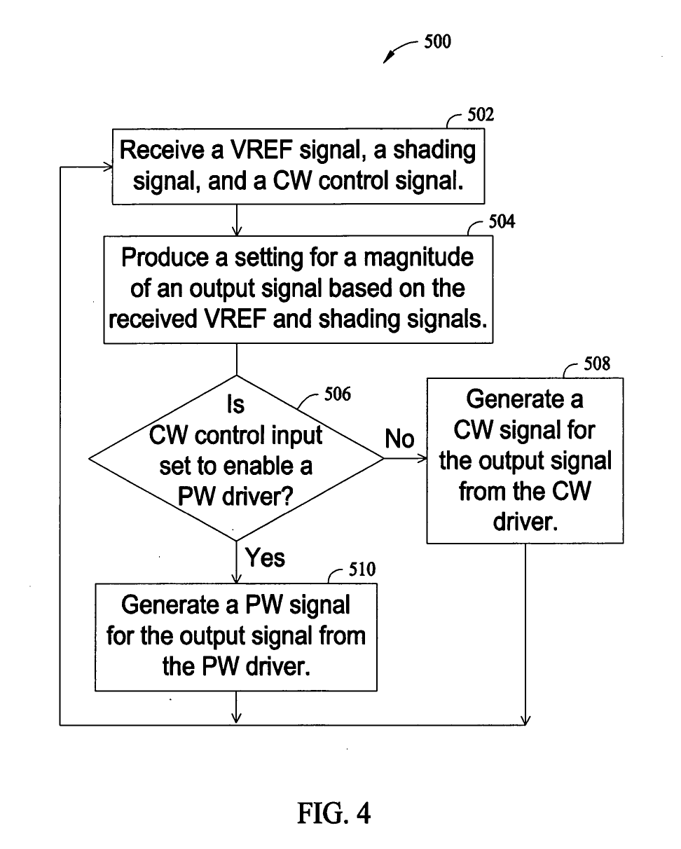

FIG. 4 is a flowchart of an exemplary method 500 of producing an output signal in accordance with an embodiment of the present invention. At 502, the pulser circuit 102 receives the reference voltage (VREF) 106, the shading signal 104, and the CW control signal 108. At 504, a pulser multiplier circuit sets a magnitude of the output signal 110 based on the VREF 106 and the shading signal 104. A determination is made at 506 whether to enable the PW driver or the CW driver of a CW-PW driver pair based on the CW control signal 108. If the CW driver is enabled, the CW driver generates at 508 the CW signal 112 for the output signal 110. If the PW driver is enabled, the PW driver generates at 510 the PW signal 114 for the output signal 110. The level or magnitude of the output signal 110 is based on the magnitude setting produced at 504 by the multiplier. The method then repeats. During iterations of the method 500, a CW signal 112 interleaved with a PW signal 114 is thereby produced at the output 111 as the output signal 110 for use by the ultrasound transducer.

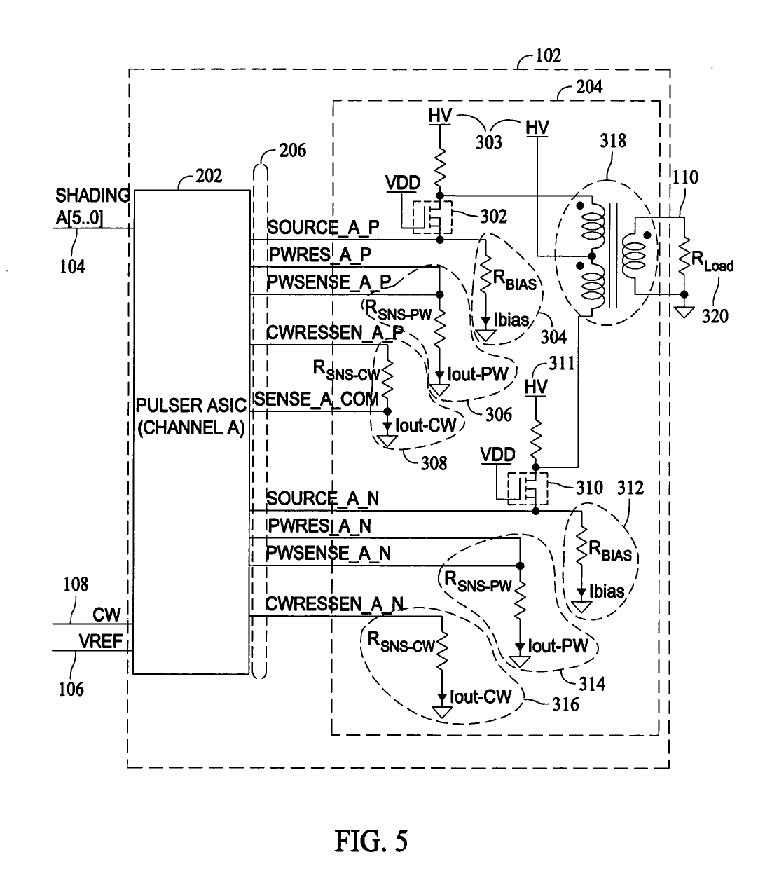

FIG. 5 is a block diagram of the FET circuitry 204 of FIG. 3. It should be noted that the interface 206 provides control signaling related to one output channel (e.g., channel A) of the ASIC 202. In general, an ASIC may provide enough processing and signaling output to drive two or more output channels, for example, two or more of the output signals 110.

A source 304 and a source 312 are coupled correspondingly through a FET 302 and a FET 310 to a transformer 318. Signals transmitted via the interface 206 generally include suffixes with either a “P” or an “N”. The suffix “P” indicates signals for use in generating the positive portions 118 and 122 (shown in FIG. 2) of the corresponding CW signal 112 and PW signal 114. The suffix “N” indicates signals for use in generating the negative portions 120 and 124 (shown in FIG. 2) of the corresponding CW signal 112 and PW signal 114. The source 304 and the FET 302 with high voltage (HV) sources 303 provide input to the transformer 318 for the positive portions 118 and 122 of the output signal 110. The source 312 and the FET 310 with a HV source 311 provide input to the transformer 318 for the negative portions 120 and 124 of the output signal 110. The transformer 318 connects to an R-load 320, which is the transducer element that receives the output signal 110.

The PW feedback 306 provides feedback signaling into the ASIC 202 for generating the positive portion 122 of the PW signal 114, and the CW feedback 308 provides feedback signaling into the ASIC 202 for generating the positive portion 118 of the CW signal 112. Similarly, the PW feedback 314 provides feedback signaling into the ASIC 202 for generating the negative portion 124 of the PW signal 114, and the CW feedback 316 provides feedback signaling into the ASIC 202 for generating the negative portion 120 of the CW signal 112.

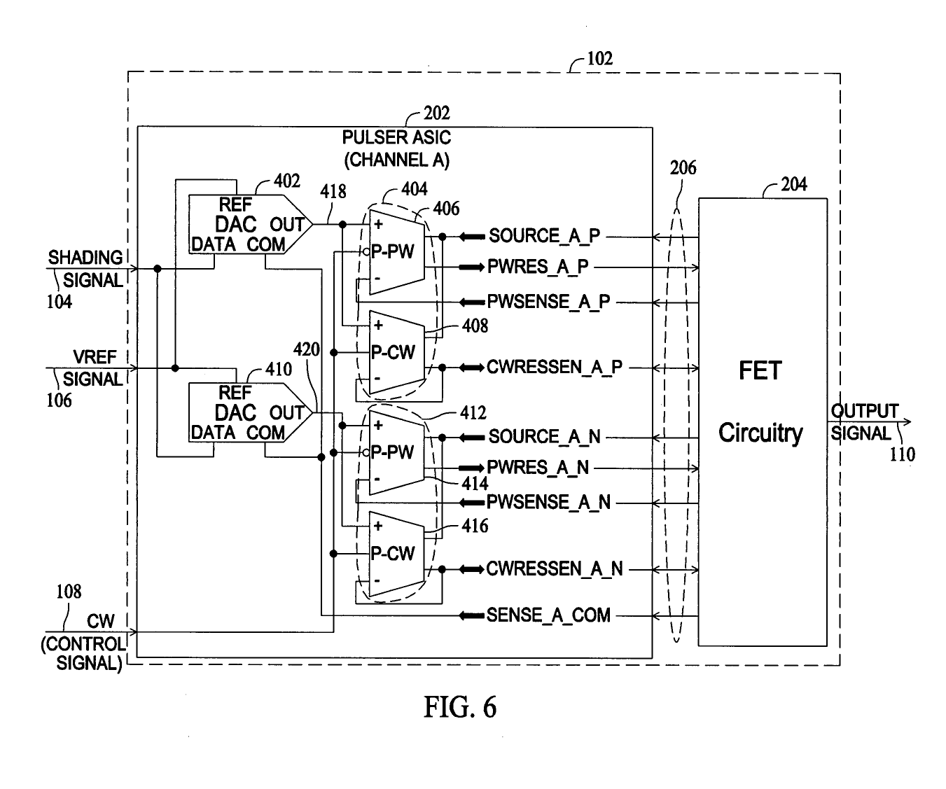

FIG. 6 is a detailed block diagram of the circuitry within the ASIC 202 of FIG. 3. A digital-to-analog converter (DAC) 402 is configured to operate as a multiplier that receives the shading signal 104 and the VREF 106 to produce a setting output 418 for use in establishing the magnitude of the output signal 110. Likewise, a DAC 410 is configured to operate as a multiplier that receives the shading signal 104 and the VREF 106 to produce a setting output 420 for use in establishing the magnitude of the output signal 110.

The setting output 418 is provided as an input to a P-PW driver 406 and a P-CW driver 408 of a driver pair 404. The output from the driver pair 404 determines the positive portions 118 and 122 of the waveform of the output signal 110. When the P-PW driver 406 is enabled, the positive portion 122 of a PW waveform 114 is generated (at 510 as shown in FIG. 4) for the output signal 110. When the P-CW driver 408 is enabled, the positive portion 118 of a CW waveform 112 is generated (at 508 as shown in FIG. 4) for the output signal 110.

Similar to the driver pair 404, a driver pair 412 of a P-PW driver 414 and a P-CW driver 416 uses the setting output 420 as an input to generate the negative portions 120 and 124 of the waveform of the output signal 110. When the P-PW driver 414 is enabled, the negative portion 124 of a PW waveform 114 is generated (at 510 as shown in FIG. 4) for the output signal 110. When the P-CW driver 416 is enabled, the negative portion 120 of a CW waveform 112 is generated (at 508 as shown in FIG. 4) for the output signal 110.

In another embodiment, the DAC 402 and DAC 410 of FIG. 5 may be replaced by linear amplifiers or other devices that perform the functionality of the DACs. In yet another embodiment, the functionality of the DAC 402 may be incorporated with the driver pair 404 as a single device. Likewise, the DAC 410 may be incorporated with the driver pair 412 as a single device.

It should be noted that ultrasound pulsers constructed in accordance with the present invention are not limited to the specific pulser embodiments described herein, and may be modified as desired or needed, for example, based on system requirements.

While the invention has been described in terms of various specific embodiments, those skilled in the art will recognize that the invention can be practiced with modification within the spirit and scope of the claims.

What is claimed is:

1. A multi-level pulser for an ultrasound system, comprising:
a first input for receiving a reference voltage signal;
a second input for receiving a shading signal;
a third input for receiving a control signal; and
an output for driving a transducer, said output producing an output signal based on said reference voltage signal, said shading signal, and said control signal, said output signal comprising a continuous wave (CW) signal interleaved with a pulse wave (PW) signal.

2. The multi-level pulser of claim 1, wherein a level of the PW signal varies based on said shading signal.

3. The multi-level pulser of claim 1, wherein said shading signal comprises a digital six bit word.

4. The multi-level pulser of claim 1, wherein said output signal comprises maximum value based on said reference voltage.

5. The multi-level pulser of claim 1, wherein said control signal is configured to be used to control the interleaving of the CW signal with the PW signal.

6. The multi-level pulser of claim 1, further comprising a CW driver and a PW driver, said control signal activating the CW driver to produce the CW signal and activating the PW driver to produce the PW signal.

7. The multi-level pulser of claim 1, further comprising a multiplier configured to multiply said reference voltage signal and said shading signal to provide a magnitude setting of said output signal to said output.

8. The multi-level pulser of claim 1, further comprising an application specific integrated circuit (ASIC) for receiving said reference voltage signal, said shading signal, and said control signal.

9. The multi-level pulser of claim 1, further comprising an ASIC interfaced with field effect transistor (FET) circuitry, the ASIC receiving said first, second, and third inputs, and the FET circuitry producing said output.

10. The multi-level pulser of claim 1, further comprising an application specific integrated circuit (ASIC) for receiving said reference voltage signal, said shading signal, and said control signal, the ASIC further comprising a multiplier for multiplying said reference voltage and said shading signal to provide a magnitude setting for the magnitude of said output signal, the magnitude setting providing input to a pair of CW and PW drivers.

11. A method for producing a pulser signal to an ultrasound system transducer, said method comprising:
receiving a reference voltage signal for a first input;
receiving a shading signal for a second input;
receiving a control signal for a third input; and
producing an output signal for driving a transducer, the output signal based on the reference voltage signal, the shading signal, and the control signal, the output signal comprising a continuous wave (CW) signal interleaved with a pulse wave (PW) signal.

12. The method of claim 11, wherein said producing an output signal further comprises varying a level of the pulse wave (PW) signal based on the shading signal.

13. The method of claim 11, wherein the shading signal comprises a digital six bit word.

14. The method of claim 11, wherein said producing an output signal further comprises setting a maximum value for the output signal based on the reference voltage signal.

15. The method of claim 11, wherein said producing an output signal further comprises controlling the interleaving of a continuous wave (CW) signal with a pulse wave (PW) signal based on the control signal.

16. The method of claim 11, wherein said producing an output signal comprises producing a continuous wave (CW) signal by a CW driver and a pulse wave (PW) signal by a PW driver, the CW signal and PW signal interleaved with one another over time.

17. The method of claim 11, wherein said producing an output signal comprises producing a positive portion and a negative portion of the output signal, the positive portion produced by a first CW-PW driver pair, the negative portion produced by a second CW-PW driver pair.

18. The method of claim 11, wherein said producing an output signal further comprises multiplying by a multiplier the reference voltage signal and the shading signal to provide a magnitude setting of the output signal.

19. The method of claim 11, wherein said producing an output signal comprises interfacing an application specific integrated circuit (ASIC) with field effect transistor (FET) circuitry, the ASIC receiving the reference voltage input signal, the shading signal, and the control signal input.

20. The method of claim 19, wherein the ASIC comprises a multiplier for multiplying the reference voltage signal and the shading signal to produce a magnitude setting, and a CW-PW driver pair receiving the magnitude setting to produce the output signal.
